(19) United States
(12) Patent Application Publication
(10) Pub. No.: US 2024/0417770 A1
(43) Pub. Date: Dec. 19, 2024

(54) mRNA Capping Enzyme And Methods of Use Thereof

(71) Applicant: Nature's Toolbox, Inc., Rio Rancho, NM (US)

(72) Inventors: Matthias Strieker, Santa Fe, NM (US); Michael Humbert, Santa Fe, NM (US); Alexander Koglin, Santa Fe, NM (US); Grant Eliason, Albuquerque, NM (US)

(21) Appl. No.: 18/755,160

(22) Filed: Jun. 26, 2024

Related U.S. Application Data
(63) Continuation-in-part of application No. PCT/US23/62161, filed on Feb. 7, 2023.
(60) Provisional application No. 63/308,165, filed on Feb. 9, 2022.

(51) Int. Cl. C12P 19/34; C12N 9/10; C12N 9/12

(52) U.S. Cl. CPC C12P 19/34; C12N 9/1007; C12N 9/1247; C12N 9/1276; C12Y 201/01102; C12Y 207/07006; C12Y 207/0705

(57) ABSTRACT

Provided herein is a method for capping RNA in vitro. In some embodiments the capping reaction may be done using an RNA guanylyltransferase capping enzyme from a Tupanviruses virus, and in particular a capping enzyme from Tupanvirus soda lake virus or Tupanvirus deep ocean virus, or a fragment or variant thereof. Compositions and kits for capping RNA in vitro are also provided. Also included are methods, compositions and kits for the manufacturer and capping of therapeutic RNA oligonucleotides, such as RNA-based vaccines and therapeutics.

### CROSS-REFERENCE TO RELATED APPLICATIONS

This International PCT Application claims the benefit of and priority to U.S. Provisional Application No. 63/308,165 filed Feb. 9, 2022. The entire specification, claims, and figures of the above-referenced application is hereby incorporated, in its entirety by reference.

### SEQUENCE LISTING

The instant application contains contents of the electronic sequence listing (90125-00241-Sequence-Listing.xml; Size: 7,213 bytes; and Date of Creation: Feb. 7, 2023) is herein incorporated by reference in its entirety.

### TECHNICAL FIELD

The present invention is directed to the production of mRNA transcripts. In particular, the novel systems and compositions to facilitate the high-efficiency addition of a cap to the 5′ end of an uncapped RNA in in vitro production systems.

### BACKGROUND

Most eukaryotic cellular mRNA transcripts and most eukaryotic viral mRNA transcripts are blocked or “capped” at their 5′ terminus. In addition to mRNA, some other forms of eukaryotic RNA, such as but not limited to, small nuclear RNA (“snRNA”) and pre-micro RNA (i.e., “pre-miRNA”, the primary transcripts that are processed to miRNA) are also capped. A “cap” is a guanine nucleoside that is joined via its 5′-carbon to a triphosphate group that is, in turn, joined to the 5′-carbon of the most 5′-nucleotide of the primary mRNA transcript, and in most eukaryotes, the nitrogen at the 7 position of guanine in the cap nucleotide is methylated. Such a capped transcript can be represented as m⁷G(5′)ppp(5′)N₁(pN)ₓ—OH (3′), or more simply, as m⁷GpppN₁ (pN)ₓ, where m⁷G represents the 7-methylguanosine cap nucleoside, ppp represents the triphosphate bridge between the 5′ carbons of the cap nucleoside and the first nucleotide of the primary RNA transcript, and N₁(pN)ₓ—OH (3′) represents the primary RNA transcript, of which N₁ is the most 5′-nucleotide. The 5′ caps of eukaryotic cellular and viral mRNAs (and some other forms of RNA) play important roles in RNA stability and processing. For example, the cap plays a pivotal role in mRNA metabolism, and is required to varying degrees for processing and maturation of an RNA transcript in the nucleus, transport of mRNA from the nucleus to the cytoplasm, mRNA stability, and efficient translation of the mRNA to protein.

In vitro, capped RNAs have been reported to be translated more efficiently than uncapped transcripts in a variety of in vitro translation systems, such as rabbit reticulocyte lysate or wheat germ translation systems. This effect is also believed to be due in part to protection of the RNA from exoribonucleases present in the in vitro production system, as well as other factors. Therefore, the importance of the cap can vary with the particular translation system and its method of preparation. Moreover, uncapped RNAs, and in particular RNAs that have a 5′-triphosphate are reported to activate the innate immune response. As such, it is highly desirable to add a cap to synthetic RNA in many therapeutic applications (e.g., protein replacement therapy as well as prophylactic or therapeutic vaccination).

Currently, only two methods are used to cap RNA. In the first method, RNAs transcribed in vitro, sometimes referred to as synthetic RNAs, are converted to capped RNAs using an RNA capping enzyme. The most common capping enzyme is tri-functional enzyme called VCE derived from the Vaccinia virus. However, the VCE enzyme is unstable and is not expressed efficiently in traditional expression systems, such as E. coli. Moreover, use of VCE is not cost effective. VCE alone currently represents 40% of the costs of a RNA vaccine. In the other method, generally referred to as “co-transcriptional capping” cap analogs such as anti-reverse cap analog (ARCA) and capped dinucleotides are added to the in vitro transcription reaction. In this system, the cap is co-transcriptionally incorporated into RNA molecules during in vitro transcription. Compared to the co-transcriptional capping method, the enzymatic RNA capping can achieve higher yields of capped RNA. However, as noted above, enzymatic RNA capping reactions are generally inefficient and, as such, either large amounts of enzyme are used, or the capped RNA must be purified from the uncapped RNA. Further, the efficiency of enzymatic RNA capping reactions (expressed as a percentage of capped RNA after the completion of a capping reaction) can vary from one RNA sequence to another, a difference that is generally attributed to RNA structure.

As such, there exists a long-felt need for a more efficient and cost-effective system to cap synthetic RNAs.

### SUMMARY OF THE INVENTION

The present invention includes systems, methods, compositions, and kits for capping RNA oligonucleotides, preferably synthetic RNAs generated in vitro. In other embodiments, the capping reactions may comprise a capping enzyme from the genus of Tupanviruses viruses, and preferably a capping enzyme from Tupanvirus soda lake virus or Tupanvirus deep ocean virus, or a fragment or variant thereof. In a preferred embodiment, the invention include systems, methods, compositions, and kits to generate the all or part of the enzymatic reactions are involved in capping of an uncapped mRNA transcript, generally including: (1) a RNA triphosphatase cleaves the 5′-triphosphate of mRNA to a diphosphate; (2) a RNA guanyltransferase catalyzes joining of GTP to the 5′-diphosphate of the most 5′ nucleotide; and (3) a guanine-7-methyltransferase, using S-adenosyl-methionine as a co-factor, catalyzes methylation of the 7-nitrogen of guanine in the cap nucleotide.

The present invention provides a method for efficiently capping RNA oligonucleotides in vitro. In some embodiments, the method may comprise contacting an RNA sample comprising an uncapped RNA with an capping enzyme of the invention comprising an amino acid sequence SEQ ID NO: 1 or 2, or a fragment or variant thereof. An uncapped RNA optionally may be free of modified nucleotides or may comprise one or more modified nucleotides such as pseudouridine.

In another embodiment, the present invention includes a method of contacting an RNA sample comprising an uncapped RNA with an capping enzyme of the invention comprising an amino acid sequence SEQ ID NO: 1 or 2, or a capping enzyme having at least 80% or at least 90% sequence identity with SEQ ID NO. 1 or 2. In a preferred embodiment, the method may be performed in an in vitro transcription system, and preferably a cell-free expression/RNA production system as described herein.

The present invention provides compositions for efficiently capping RNA oligonucleotides in vitro. In some embodiments, the composition of the invention includes a capping enzyme comprising an amino acid sequence SEQ ID NO: 1 or 2, or a fragment or variant thereof. In another embodiment, the composition of the invention includes a capping enzyme comprising an amino acid sequence SEQ ID NO: 1 or 2, or a capping enzyme having at least 80% or at least 90% sequence identity with SEQ ID NO. 1 or 2. In a preferred embodiment, composition may be performed in an in vitro transcription system, and preferably a cell-free expression/RNA production system as described herein. The capping enzyme of the invention may be isolated, for example through the addition of a tag, such as a His-tag according to SEQ ID NO. 3, to allow isolation through affinity chromatography or other protein isolation and purification techniques known in the art.

The present invention provides an expression vector encoding a capping enzyme, or a fragment or variant thereof. In this embodiment, the expression vector may include an expression cassette encoding a nucleotide sequence, operably linked to a promoter, that generates a capping enzyme comprising an amino acid sequence SEQ ID NO: 1, or a fragment or variant thereof. In this embodiment, the may include an expression cassette encoding a nucleotide sequence, operably linked to a promoter, that generates a capping enzyme comprising an amino acid sequence SEQ ID NO: 1, or a nucleotide sequence that generates a capping enzyme having at least 80% with SEQ ID NO. 1 or 2, or at least 90% sequence identity with SEQ ID NO. 1 or 2. In another embodiment, the expression vector of the invention may be heterologously expressed in a cell, such a prokaryotic or eukaryotic cell and further isolated as noted above.

The present invention also comprises compositions comprising the modified-nucleotide-capped RNA made using kits and methods of the present invention. For example, composition are provided that are made using a kit comprising a capping enzyme and a modified cap nucleotide or a composition made using the method comprising contacting an uncapped RNA comprising a primary RNA transcript or an RNA having a 5′-diphosphate with a modified cap nucleotide and a capping enzyme system, wherein a modified-nucleotide-capped RNA is synthesized. Thus, the present invention includes new compositions of modified-nucleotide-capped RNA not previously known in the art.

Additional aspects of the invention will be apparent to one of ordinary skill in the art from the specification, figures and claims provided below.

### BRIEF DESCRIPTION OF THE FIGURES

FIG. 1: shows a general schematic process for the in vitro production of mRNA oligonucleotides, including the steps of in vitro transcription from a DNA template, capping of the RNA transcript followed by methylation of the cap.

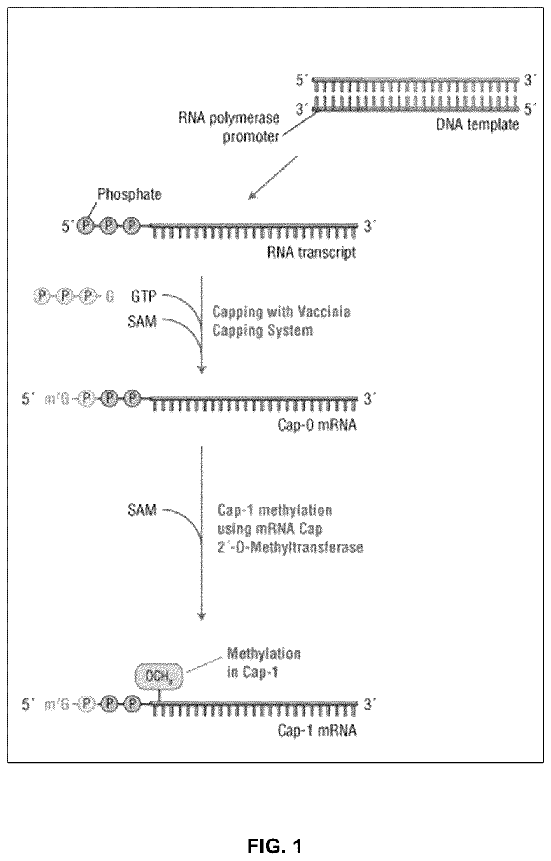

FIG. 2: shows a general schematic process for the in vitro production of mRNA oligonucleotides, including polyadenylation of the mRNA transcript.

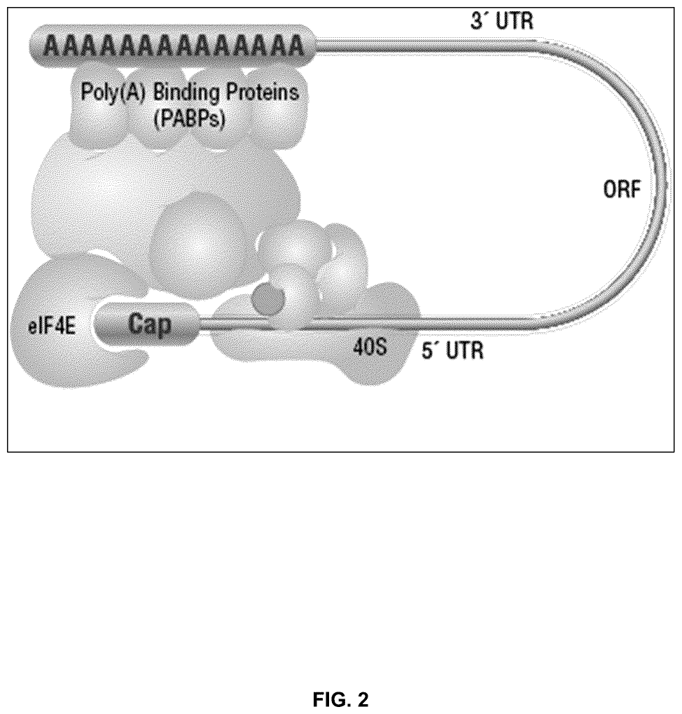

FIG. 3: shows a schematic map of a plasmid expression vector that expresses the capping enzyme of the invention in one embodiment thereof.

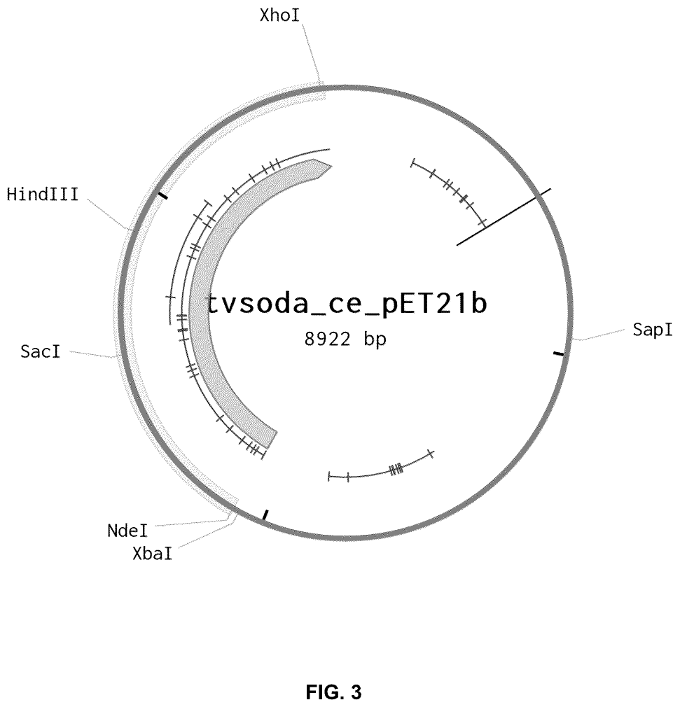

FIG. 4: shows confirmation of the expression of the vector generating the capping enzyme of the invention, which in this embodiment include a His-tag for isolation in one embodiment thereof.

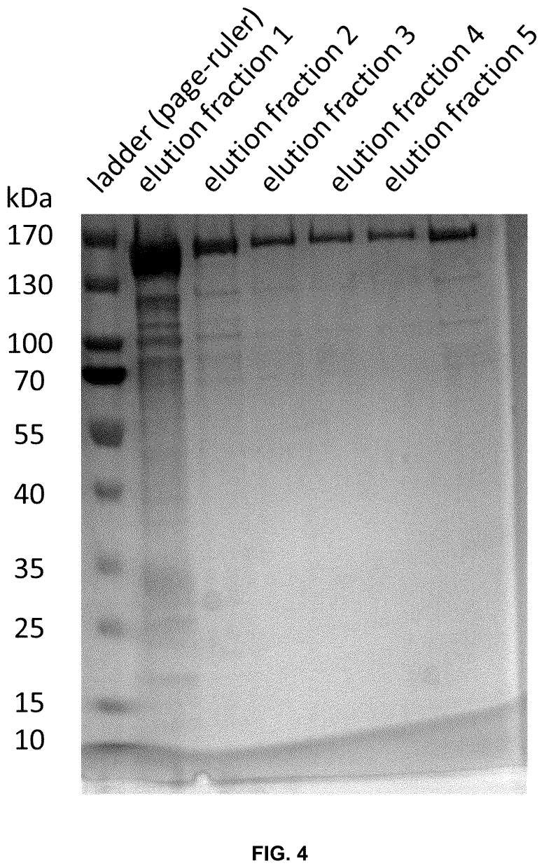

FIG. 5A-B: shows the characterization of the activity of the capping enzyme of the invention. (A) bioanalyzer data demonstrating an unmodified (uncapped) mRNA transcript; (B) bioanalyzer data demonstrating the characterization of the activity of the capping enzyme of the invention incubated with an exemplary 15-mer with 1:10 tupanvirus capping enzyme: mRNA, and showing approximately 46-88% conversion (capping) after 30 minutes.

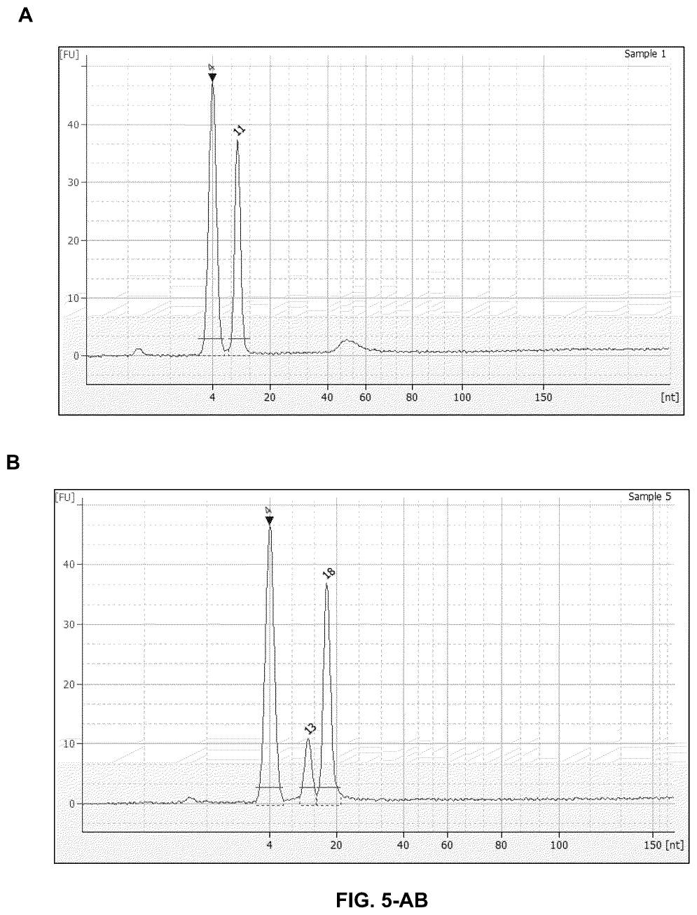

FIG. 6: shows the characterization of the activity of the capping enzyme of the invention by the percent of capped transcripts by incubation with varying molar ratios of enzyme: mRNA, determined with area under the peak values from bioanalyzer analysis.

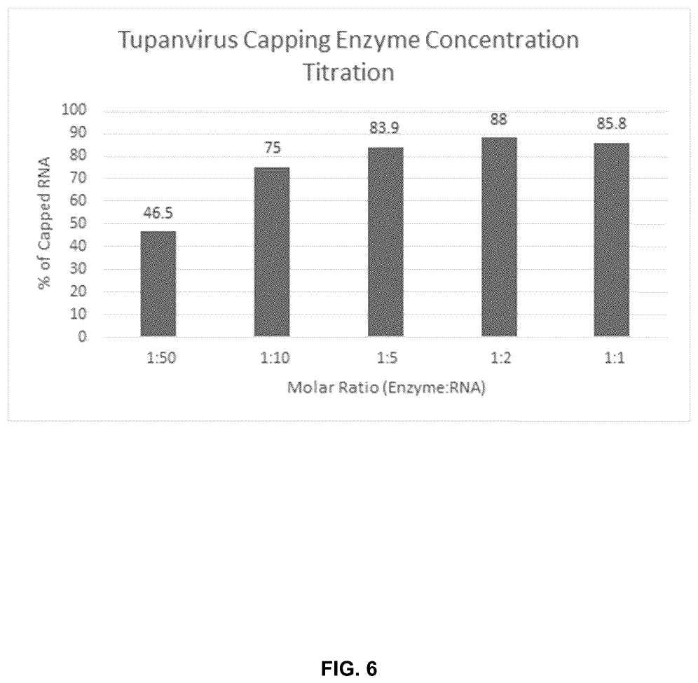

FIG. 7: shows the characterization of the activity of the capping enzyme of the invention by the percent of capped transcripts by incubation of enzyme and RNA transcripts at varying temperatures, determined with area under the peak values from bioanalyzer analysis.

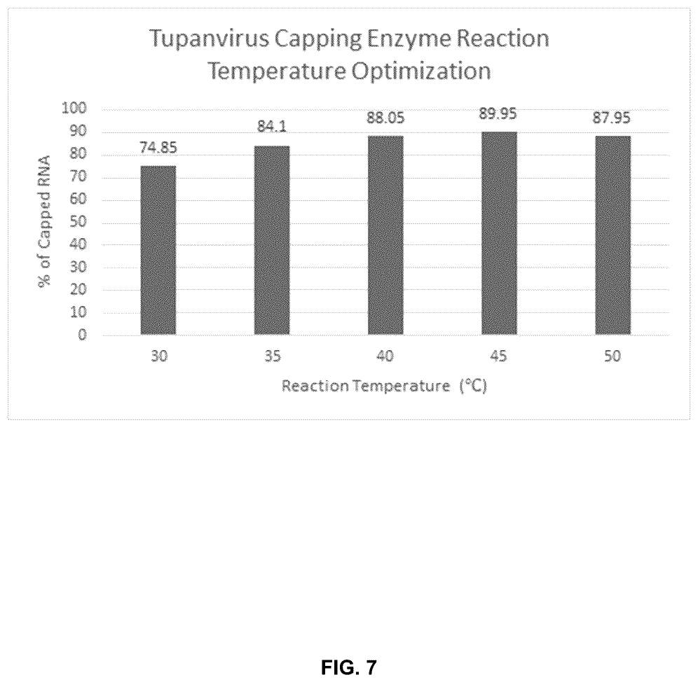

FIG. 8: shows a time-course characterization of the activity of the capping enzyme of the invention by the percent of capped transcripts by incubation with a 1:5 enzyme to RNA ratio at an exemplary 42° C., determined with area under the peak values from bioanalyzer analysis. Additional assays run at 40° C. and 45° C. (not shown)

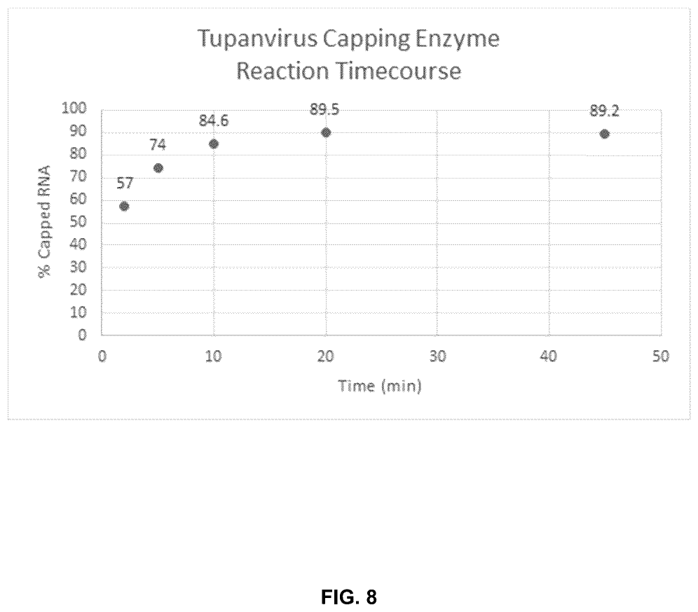

FIG. 9: shows expression and controls for heterologous expression of capping enzyme in HEK-293T cells resulting in in vivo capping of mRNA

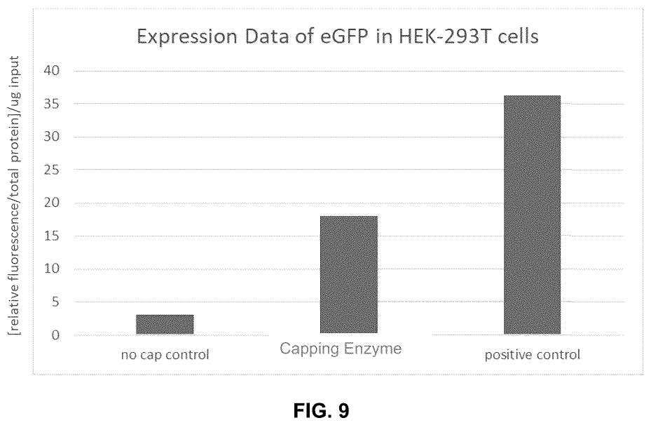

### DETAILED DESCRIPTION OF THE INVENTION

In one aspect, the inventive technology includes systems, methods and compositions for capping RNA oligonucleotides, and preferably capping RNA oligonucleotides in vitro. In additional aspects, the invention may comprise methods and compositions including the RNA sample comprising an uncapped target RNA that can be contacted with an RNA guanylyltransferase capping enzyme from a Tupanviruses virus, or a fragment or variant thereof, under conditions wherein a Cap-0 RNA is synthesized in vitro. The said capping enzyme from a Tupanviruses virus may include a capping enzyme from Tupanvirus soda lake virus or Tupanvirus deep ocean virus. In a preferred aspect, RNA capping enzyme comprising an amino acid sequence that is at least 90% identical to SEQ ID NO: 1. The RNA capping enzyme may further include a tag, such as a His-6 tag to facilitate isolation and purification according to SEQ ID NO. 3. In other aspects, guanosine triphosphate (GTP) or modified GTP, and a buffering agent may also be contacted to facilitate the synthesis of Cap-0 RNA is synthesized in vitro.

In still further aspects of the invention, the method may include methylating the Cap-0 RNA forming a Cap-1 RNA. In this embodiment, the Cap-0 RNA may be contacted with methyltransferase, such as a guanine-7-methyltransferase, and a methyl donor, such as S-adenosyl methionine (SAM). An exemplary SAM being provided at Accession No. AAG58487, or a sequence having at least 80% sequence identity.) The efficiency determined by yield of capped RNA may be improved by adjusting the temperature and ratio of capping enzyme and uncapped RNA oligonucleotides. In this embodiment, the ratio of RNA guanylyltransferase capping enzyme and uncapped RNA may be between 1:50 and 1:1, or between 1:100 and 50:1, and the temperature of the capping reaction may be between 30° and 50° C., or between 20° and 60° C. An uncapped target RNA optionally may be free of modified nucleotides or may comprise one or more modified nucleotides (e.g., pseudouridine).

The uncapped target RNA of the invention may include uncapped RNA synthesized using solid-phase oligonucleotide synthesis chemistry, or by contacting a DNA template encoding the uncapped RNA and a polymerase (e.g., T7 RNA polymerase or Hi-T7 RNA polymerase) in an in vitro transcription reaction, to produce the uncapped RNA, which may be further modified, for example by polyadenylation. Uncapped RNA of the invention may also specifically include a primary RNA transcript or an RNA that has a 5′-diphosphate is selected from the group consisting of: prokaryotic mRNA; uncapped eukaryotic primary mRNA; RNA from an in vitro transcription reaction using an RNA polymerase; RNA from an in vitro replication reaction using a replicase; RNA from an in vivo transcription reaction, wherein the RNA polymerase is expressed in a prokaryotic or eukaryotic cell that contains a DNA template that is functionally joined downstream of an RNA polymerase promoter that binds the RNA polymerase; RNA from an in vivo replication reaction using a replicase; RNA from an RNA amplification reaction; eukaryotic small nuclear (snRNA); and micro RNA (miRNA) and the like.

In some embodiments, an uncapped target RNA may be less than 200 nt in length, at least 200 nt in length (e.g., at least 300 nt, at least 500 nt or at least 1,000 nt) and may encode a polypeptide such as a therapeutic protein or vaccine. Target RNA having secondary structure including therapeutic RNA can be capped more efficiently using method of this disclosure. The capping method and compositions described herein, in some embodiments, may produce (e.g., may co-transcriptionally produce) approximately 90% Cap-0 RNA in vitro in thirty-minutes or less. In some embodiments, the components and/or combinations thereof may be RNase-free and contacting may optionally further comprise one or more RNase inhibitors.

The production of Cap-0 RNA from uncapped target RNA oligonucleotides may be performed in vitro, and in particular in a bioreactor, a microfluidics surface, a reaction tube or other reaction vessel configured to produce mRNA or other macromolecules such as proteins for therapeutic or industrial purposes. Examples may include in vitro transcription systems, cell-free expression systems as generally described or incorporated herein.

Also provided herein is a composition comprising an uncapped target RNA; and a RNA guanylyltransferase capping enzyme from a Tupanviruses virus, or a fragment or variant thereof, guanosine triphosphate (GTP), and a buffering agent. The capping enzyme from a Tupanviruses virus may include a capping enzyme from Tupanvirus soda lake virus or Tupanvirus deep ocean virus. In a preferred aspect, RNA capping enzyme comprising an amino acid sequence that is at least 90% identical to SEQ ID NO: 1. The RNA capping enzyme may further include a tag, such as a His-6 tag to facilitate isolation and purification according to SEQ ID NO. 3. The composition of the invention may further include a DNA template polymerase, such as a T7 bacteriophage polymerase and ribonucleotides, for transcribing a template polynucleotide encoding a uncapped target RNA, as well as a methyltransferase, such as a guanine-7-methyltransferase or 2′-O-methyltransferase enzyme, and a methyl donor, such as S-adenosyl methionine (SAM). In some aspects, a composition may be RNase-free and may optionally comprise one or more RNase inhibitors. In some embodiments, a composition may further comprise a DNA template, a polymerase (e.g., a bacteriophage polymerase) and ribonucleotides, for transcribing the DNA template to form the uncapped target RNA. In some embodiments, a single uncapped target RNA may be at least 200 nt in length (at least 300 nt, at least 500 nt or at least 1,000 nt) and may encode a polypeptide such as a therapeutic protein or vaccine.

Additional aspects may include a kit for producing capped RNA, and preferably mRNA transcripts in vitro. In some embodiments, the kit of the invention may include an RNA guanylyltransferase capping enzyme from a Tupanviruses virus, or a fragment or variant thereof, wherein the enzyme is in a storage buffering agent; and a reaction buffering agent. Additional aspects may include a kit for producing capped RNA, and preferably mRNA transcripts in a cell-free expression system comprising, a reaction mixture having cell-free reaction components necessary for in vitro macromolecule synthesis, including an RNA guanylyltransferase capping enzyme from a Tupanviruses virus, or a fragment or variant thereof; and a storage buffering agent. The capping enzyme from a Tupanviruses virus may include a capping enzyme from Tupanvirus soda lake virus or Tupanvirus deep ocean virus. In a preferred aspect, RNA capping enzyme comprising an amino acid sequence that is at least 90% identical to SEQ ID NO. 1 or 2. The RNA capping enzyme may further include a tag, such as a His-6 tag to facilitate isolation and purification according to SEQ ID NO. 3.

The kit of the invention may further include a DNA template polymerase, such as a T7 bacteriophage polymerase and ribonucleotides, for transcribing a template polynucleotide encoding a uncapped target RNA, as well as a methyltransferase, such as a guanine-7-methyltransferase or 2′-O-methyltransferase enzyme, and a methyl donor, such as S-adenosyl methionine (SAM). In some aspects, a kit may be RNase-free and may optionally comprise one or more RNase inhibitors. In some embodiments, a kit may further comprise a DNA template, a polymerase (e.g., a bacteriophage polymerase) and ribonucleotides, for transcribing the DNA template to form the uncapped target RNA. In some embodiments, a single uncapped target RNA may be at least 200 nt in length (at least 300 nt, at least 500 nt or at least 1,000 nt) and may encode a polypeptide such as a therapeutic protein or vaccine.

In some embodiments, an RNA capping enzyme may include an a peptide having an amino acid sequence according to SEQ ID NO: 1 or 2, which may preferably be isolated. In some embodiments, an RNA capping enzyme may include an amino acid sequence that is at least 90% identical to SEQ ID NO: 1 or 2. In other embodiments, an RNA capping enzyme may include an amino acid sequence that is at least 80% identical to SEQ ID NO: 1 or 2. In some embodiments, an expression vector having a nucleotide sequence, operably linked to a promoter, encoding an RNA capping enzyme comprises an amino acid sequence according to SEQ ID NO: 1 or 2. In other embodiments, an expression vector having a nucleotide sequence, operably linked to a promoter, encoding an RNA capping enzyme comprises an amino acid sequence that is at least 80% identical to SEQ ID NO: 1 or 2. In other embodiments, an expression vector having a nucleotide sequence, operably linked to a promoter, encoding an RNA capping enzyme comprises an amino acid sequence that is at least 90% identical to SEQ ID NO: 1 or 2.

In one aspect, the invention provides for efficient capping of RNA substrate and may support capping with less enzyme added to a capping reaction, producing more capped RNA (as a percentage of the RNA in the reaction) using the same amount of enzyme, terminating the reaction earlier, and/or capping RNAs that have secondary structure at the 5′ end more efficiently. Notably, Disclosed reaction conditions may be varied, including, without limitation, reaction temperature, reaction duration, reaction component concentrations (e.g., SAM, inorganic pyrophosphatase, NTPs, transcript template), and enzymes (e.g., capping enzymes, polymerases, and etc). Depending on which enzyme is used and the other components in the reaction mix, disclosed methods may be used to make RNAs that have a Gppp cap, a 7-methylguanylate cap (i.e., a m⁷Gppp cap, or “cap-0”), or an RNA that has an m⁷Gppp cap that has addition modifications in the first and/or second nucleotides in the RNA (i.e., “cap-1” and “cap-2”; see Fechter J. Gen. Vir. 2005 86:1239-49). For example, if there is no SAM in the reaction mix then RNAs that have a Gppp cap may be produced. If the reaction mix comprises SAM in addition to the capping enzyme, then cap-0 RNA may be produced. If the reaction mix comprises other enzymes, e.g., cap 2′OMTase, such as an exemplary Vaccinia virus cap 2′OMTase (New England Biolabs, Ipswich, Mass.), in addition to SAM, then cap-1 and/or cap-2 RNA may be produced. A reaction mix may comprise other components in addition to those explicitly described above.

In some embodiments, uncapped RNA in the reaction mix may be prepared by solid-phase oligonucleotide synthesis chemistry (see, e.g., Li, et al J. Org. Chem. 2012 77:9889-9892), in which case the RNA in the sample may be may have a length in the range of 10-500 bases, e.g., 20-200 bases). In other embodiments, uncapped RNA in the reaction mix may be prepared in a cell free in vitro transcription (IVT) reaction in which a double-stranded DNA template that contains a promoter for an RNA polymerase (e.g., a T7, T3 or SP6 promoter) upstream of the region that is transcribed is copied by a DNA-directed RNA polymerase (typically a bacteriophage polymerase) to produce a product that contains RNA molecules copied from the template. In either embodiment, the RNA sample that is capped in the present method contains a single species of RNA (either the synthetic oligonucleotide or the transcript). In addition, the reaction mix may be RNase-free and may optionally comprise one or more RNase inhibitors. The RNA in the sample may contain a non-natural sequence of nucleotides and in some embodiments, may contain non-naturally occurring nucleotides. In some embodiments, the in vitro transcription reaction may employ a thermostable variant of the T7, T3 and SP6 RNA polymerase (see, e.g., PCT/US2017/013179 and U.S. application Ser. No. 15/594,090). In these embodiments, the RNAs may be transcribed at a temperature of greater than 44° C. (e.g., a temperature of at least 45° C., at least 50° C., at least 55° C. or at least 60° C., up to about 70° C. or 75° C.) in order to reduce the immunogenicity of the RNA (see, e.g., WO 2018/236617). In some cases, the uncapped RNA may be capped immediately after it is made, e.g., by adding an RNA capping enzyme and GTP/modified GTP, as needed to the in vitro transcription reaction after the reaction has run its course. In some embodiments, the RNA made in an in vitro transcription reaction may purified prior to capping.

In some embodiments, the RNA in the sample may be a therapeutic RNA, such as a vaccine. In these embodiments, the product of the present method may be used without purification of the capped RNA from the uncapped RNA. In these embodiments, the product of the present reaction may be combined with a pharmaceutical acceptable excipient to produce a formulation, where “pharmaceutical acceptable excipient” is any solvent or composition that is compatible with administration to a living mammalian organism via transdermal, oral, intravenous, or other administration means used in the art. Examples of pharmaceutical acceptable excipients include those described for example in US 2017/0119740. The formulation may be administered in vivo, for example, to a subject, examples of which include a human or any non-human animal (e.g., mouse, rat, rabbit, dog, cat, cattle, swine, sheep, horse or primate). Depending on the subject, the RNA (modified or unmodified) can be introduced into the cell directly by injecting the RNA or indirectly via the surrounding medium. Administration can be performed by standardized methods. The RNA can either be naked or formulated in a suitable form for administration to a subject, e.g., a human. Formulations can include liquid formulations (solutions, suspensions, dispersions), topical formulations (gels, ointments, drops, creams), liposomal formulations (such as those described in: U.S. Pat. No. 9,629,804 B2; US 2012/0251618 A1; WO 2014/152211; US 2016/0038432 A1). The cells into which the RNA product is introduced may be in vitro (i.e., cells that cultured in vitro on a synthetic medium). Accordingly, the RNA product may be transfected into the cells. The cells into which the RNA product is introduced may be in vivo (cells that are part of a mammal). Accordingly, the introducing may be done by administering the RNA product to a subject in vivo. The cells into which the RNA product is introduced may be present ex vivo (cells that are part of a tissue, e.g., a soft tissue that has been removed from a mammal or isolated from the blood of a mammal).

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference. Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins. It is further noted that the claims can be drafted to exclude any optional element.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

As used herein, “buffering agent”, refers to an agent that allows a solution to resist changes in pH when acid or alkali is added to the solution. Examples of suitable non-naturally occurring buffering agents that may be used in the compositions, kits, and methods of the invention include, for example, Tris, HEPES, TAPS, MOPS, tricine, or MES. As used herein, “reaction buffering agent”, As used herein, “reaction buffer” refers to a buffer solution in which an enzymatic reaction is performed.

As used herein, “Tupanvirus” refers to viruses belonging to the genus or large viruses Tupanvirus, pacifistically including the species Tupanvirus deep ocean and Tupanvirus soda lake.

As used herein, “capping” refers to the enzymatic addition of a Nppp-moiety onto the 5′ end of an RNA, where N a nucleotide such as is G or a modified G. A modified G may have a methyl group at the N7 position of the guanine ring, or an added label at the 2 or 3 position of the ribose, and, in some embodiments, the label may be an oligonucleotide, a detectable label such as a fluorophore, or a capture moiety such as biotin or desthiobiotin, where the label may be optionally linked to the ribose of the nucleotide by a linker, for example. See, e.g., WO 2015/085142. A cap may have a Cap-O structure, a Cap-1 structure or a Cap-2 structure (as reviewed in Ramanathan, Nucleic Acids Res. 2016 44:7511-7526), depending on which enzymes and/or whether SAM is present in the capping reaction. As used herein, a “5′-cap” describes: A 5′-cap structure that is typically a modified nucleotide (cap analogue), particularly a guanine nucleotide, added to the 5′-end of an mRNA molecule. Preferably, the 5′-cap is added using a 5′-5′-triphosphate linkage (also named m⁷GpppN). Further examples of 5′-cap structures include glyceryl, inverted deoxy abasic residue (moiety), 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′ phosphorothioate, phosphorodithioate, or bridging or non-bridging methylphosphonate moiety. These modified 5-cap structures may be used in the context of the present invention to modify the mRNA sequence of the inventive composition. Further modified 5-cap structures which may be used in the context of the present invention are CAP1 (additional methylation of the ribose of the adjacent nucleotide of m⁷GpppN), CAP2 (additional methylation of the ribose of the 2nd nucleotide downstream of the m⁷GpppN), cap3 (additional methylation of the ribose of the 3rd nucleotide downstream of the m⁷GpppN), capz (additional methylation of the ribose of the 4th nucleotide downstream of the m⁷GpppN), ARCA (anti-reverse CAP analogue), modified ARCA (e.g. phosphothioate modified ARCA), inosine, N1-methyl-guanosine, 2 ‘-fluoro-guanosine, 7-deaza-guanosine, 8-oxo-guanosine, 2-amino-guanosine, LNA-guanosine, and 2-azido-guanosine. As used herein, “DNA template” refers to a double stranded DNA molecule that is transcribed in an in vitro transcription reaction. DNA templates have a promoter (e.g., a T7, T3 or SP6 promoter) recognized by the RNA polymerase upstream of the region that is transcribed.

Polyadenylation is typically understood to be the addition of a poly (A) sequence to a nucleic acid molecule, such as an RNA molecule, e.g., to a premature mRNA. Polyadenylation may be induced by a so called polyadenylation signal. This signal is preferably located within a stretch of nucleotides at the 3’-end of a nucleic acid molecule, such as an RNA molecule, to be polyadenylated. A polyadenylation signal typically comprises a hexamer consisting of adenine and uracil/thymine nucleotides, preferably the hexamer sequence AAUAAA. Other sequences, preferably hexamer sequences, are also conceivable. Polyadenylation typically occurs during processing of a pre-mRNA (also called premature-mRNA). Typically, RNA maturation (from pre-mRNA to mature mRNA) comprises the step of polyadenylation. 5′-cap structure: A 5′-cap is typically a modified nucleotide (cap analogue), particularly a guanine nucleotide, added to the 5′-end of an mRNA molecule. Preferably, the 5′-cap is added using a 5′-5′-triphosphate linkage (also named m⁷GpppN). Further examples of 5′-cap structures include glyceryl, inverted deoxy abasic residue (moiety), ‘,5’ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′ phosphorothioate, phosphorodithioate, or bridging or non-bridging methylphosphonate moiety. These modified 5-cap structures may be used in the context of the present invention to modify the mRNA sequence of the inventive composition. Further modified 5′-cap structures which may be used in the context of the present invention are CAP1 (additional methylation of the ribose of the adjacent nucleotide of m⁷GpppN), CAP2 (additional methylation of the ribose of the 2nd nucleotide downstream of the m⁷GpppN), cap3 (additional methylation of the ribose of the 3rd nucleotide downstream of the m⁷GpppN), capz (additional methylation of the ribose of the 4th nucleotide downstream of the m⁷GpppN), ARCA (anti-reverse CAP analogue), modified ARCA (e.g. phosphothioate modified ARCA), inosine, N1-methyl-guanosine, 2-fluoro-guanosine, 7-deaza-guanosine, 8-oxo-guanosine, 2-amino-guanosine, LNA-guanosine, and 2-azido-guanosine.

A vaccine is typically understood to be a prophylactic or therapeutic material providing at least one antigen or antigenic function. The antigen or antigenic function may stimulate the body's adaptive immune system to provide an adaptive immune response. An antigen-providing mRNA in the context of the invention may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g., a fusion protein that consist of two or more epitopes, peptides or proteins derived from the same or different virus-proteins, wherein the epitopes, peptides or proteins may be linked by linker sequences.

As used herein, “in vitro transcription” (IVT) refers to a cell-free reaction in which a double-stranded DNA (dsDNA) template is copied by a DNA-directed RNA polymerase (typically a bacteriophage polymerase) to produce a product that contains RNA molecules copied from the template. An example of in vitro transcription may include cell-free expression systems that produce RNA transcripts or other macromolecules, such as peptides. In certain embodiments, the invention may encompass the in vitro production of artificial mRNA as well as wild-type mRNA. An artificial mRNA (sequence) may typically be understood to be an mRNA molecule, that does not occur naturally. In other words, an artificial mRNA molecule may be understood as a non-natural mRNA molecule. Such mRNA molecule may be non-natural due to its individual sequence (which does not occur naturally) and/or due to other modifications, e.g., structural modifications of nucleotides which do not occur naturally. Typically, artificial mRNA molecules may be designed and/or generated by genetic engineering methods to correspond to a desired artificial sequence of nucleotides (heterologous sequence). In this context an artificial sequence is usually a sequence that may not occur naturally, i.e., it differs from the wild type sequence by at least one nucleotide. The term “wild type” may be understood as a sequence occurring in nature. Further, the term “artificial nucleic acid molecule” is not restricted to mean “one single molecule” but is, typically, understood to comprise an ensemble of identical molecules. Accordingly, it may relate to a plurality of identical molecules contained in an aliquot.

In certain embodiment, the invention may encompass the in vitro production of bi-/multicistronic mRNA: mRNA, that typically may have two (bicistronic) or more (multi cistronic) open reading frames (ORF) (coding regions or coding sequences). An open reading frame in this context is a sequence of several nucleotide triplets (codons) that can be translated into a peptide or protein. Translation of such an mRNA yields two (bicistronic) or more (multi cistronic) distinct translation products (provided the ORFs are not identical). For expression in eukaryotes such mRNAs may for example comprise an internal ribosomal entry site (IRES) sequence.

In one embodiment, the in vitro produce mRNA configured to be translated to form a peptide, and preferably in a host organism, such as a mammal or human subject in need thereof. A peptide is a polymer of amino acid monomers. Usually, the monomers are linked by peptide bonds. The term “peptide” does not limit the length of the polymer chain of amino acids. In some embodiments of the present invention a peptide may for example contain less than 50 monomer units. Longer peptides are also called polypeptides, typically having 50 to 600 monomeric units, more specifically 50 to 300 monomeric units.

Additional examples of IVT systems, include in vitro recombinant cell-free expression systems, which refers to the cell-free synthesis of polypeptides in a reaction mixture or solution comprising biological extracts and/or defined cell-free reaction components, such as the exemplary system described by Koglin et al., in PCT/US2020/028005 and PCT/US2021/027774 (incorporated herein by reference). The reaction mixture may optionally comprise a template, or genetic template, for production of the macromolecule, e.g., DNA, mRNA, etc.; monomers for the macromolecule to be synthesized, e.g., amino acids, nucleotides, etc.; and such co-factors, enzymes and other reagents that are necessary for the synthesis, e.g., ribosomes, tRNA, polymerases, transcriptional factors, etc. The recombinant cell-free synthesis reaction, and/or cellular adenosine triphosphate (ATP) energy regeneration system components, incorporated by reference herein, may be performed/added as batch, continuous flow, or semi-continuous flow.

Examples of in vitro production systems have been also previously described in the art, including U.S. Pat. No. 11,136,586, and PCT Application No. PCT/US2020/028005. The disclosed methods and conditions for the production of synthetic RNAs in each of the aforementioned references, including the methods, systems and compositions outline in the claims, Examples and Materials and Methods is hereby incorporated in their entirety by reference. For example, a synthetic RNA oligonucleotide may be generated and capped within a Cell Free (CF) expression system. In one embodiment, a CF expression system may include a fully recombinant stable, reliable and functional in vitro transcription system for the continuous flow production of RNA. As noted above, an exemplary CF system being generally described by A. Koglin and M. Humbert et al., in PCT/US2018/0121121, and PCT/US2021/027774 (previously identified as incorporated by reference) may be used as an in vitro platform to produce synthetic mRNAs. As noted in the art, lysate-based in vitro systems are challenged by limited stability of typical E. coli enzymes, by the activity of most metabolic processes (nucleotide recycling) and the presence of nucleases and proteases and insufficient ATP regeneration. Utilizing the CF system described by Koglin and Humbert, and using only components: linear DNA template, an affinity-tagged RNA polymerase, nucleotides in a defined buffer system, and a capping enzyme of the invention, the in vitro synthesis of the mRNA may be performed in hollow fiber reactors using a continuous flow system, as well as other traditional bioreactors known in the art. Using this in vitro system, the inner chamber (hollow fibers) of the bioreactor provides additional nucleotides in flow, the outer chamber holds the RNA polymerase and each linear DNA template. In this setup, the present inventors demonstrate that the total turnover of the RNAP is at least 50 fold higher than in batch reaction and, coupled with modifications to selected enzyme, produce cleaner mRNA without smear. All enzymes are engineered with an affinity tag, to allow the whole reaction to be washed through a bed of affinity resin, which is partially loaded with DNase to remove the template and to capture the RNA polymerase. In this way the process avoids the need to address phenol precipitations and spin column purifications (which is still an issue with traditional vaccine processes). In this system, the leaching or carryover of components from the RNA biosynthesis may be the mid ppb range. After a scalable and simple precipitation and drying process, the resulting mRNA is stable as a powder and does not contain traces of any components from the manufacturing process. It is ready to ship requiring only reduced volumes without the needed of hard-to-monitor and expensive shipping conditions.

The terms “isolated”, “purified”, or “biologically pure” as used herein, refer to material that is substantially or essentially free from components that normally accompany the material in its native state or when the material is produced. In an exemplary embodiment, purity and homogeneity are determined using analytical chemistry techniques such as polyacrylamide gel electrophoresis or high-performance liquid chromatography. A nucleic acid or particular bacteria that are the predominant species present in a preparation is substantially purified. In an exemplary embodiment, the term “purified” denotes that a nucleic acid or protein that gives rise to essentially one band in an electrophoretic gel. Typically, isolated nucleic acids or proteins have a level of purity expressed as a range. The lower end of the range of purity for the component is about 60%, about 70% or about 80% and the upper end of the range of purity is about 70%, about 80%, about 90% or more than about 90%.

In preferred embodiments, the output of the cell-free expression system may be a product, RNA, or other macromolecule such as a peptide or fragment thereof that may be isolated or purified. In this embodiment, solation or purification of a of a target protein wherein the target protein is at least partially separated from at least one other component in the reaction mixture, for example, by organic solvent precipitation, such as methanol, ethanol or acetone precipitation, organic or inorganic salt precipitation such as trichloroacetic acid (TCA) or ammonium sulfate precipitation, nonionic polymer precipitation such as polyethylene glycol (PEG) precipitation, pH precipitation, temperature precipitation, immunoprecipitation, chromatographic separation such as adsorption, ion-exchange, affinity and gel exclusion chromatography, chromatofocusing, isoelectric focusing, high performance liquid chromatography (HPLC), gel electrophoresis, dialysis, microfiltration, and the like.

he term “nucleic acid” as used herein refers to a polymer of ribonucleotides or deoxyribonucleotides. Typically, “nucleic acid” polymers occur in either single- or double-stranded form but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, and peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein. For nucleic acids, sizes are given in either kilobases (kb) or base pairs (bp). Estimates are typically derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system. All nucleotide sequences described in the invention may be codon optimized for expression in a particular organism, or for increases in production yield. Codon optimization generally improves the protein expression by increasing the translational efficiency of a gene of interest. The functionality of a gene may also be increased by optimizing codon usage within the custom designed gene. In codon optimization embodiments, a codon of low frequency in a species may be replaced by a codon with high frequency, for example, a codon UUA of low frequency may be replaced by a codon CUG of high frequency for leucine. Codon optimization may increase mRNA stability and therefore modify the rate of protein translation or protein folding. Further, codon optimization may customize transcriptional and translational control, modify ribosome binding sites, or stabilize mRNA degradation sites.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). In addition to the degenerate nature of the nucleotide codons which encode amino acids, alterations in a polynucleotide that result in the production of a chemically equivalent amino acid at a given site, but do not affect the functional properties of the encoded polypeptide, are well known in the art. “Conservative amino acid substitutions” are those substitutions that are predicted to interfere least with the properties of the reference polypeptide. In other words, conservative amino acid substitutions substantially conserve the structure and the function of the reference protein. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide. Exemplary conservative amino acid substitutions are known by those of ordinary skill in the art. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Further disclosure of a nucleotide sequence, specifically includes the resulting amino acid sequence for which it encodes and vice versa.

Homology, or sequence identity (e.g., percent homology, sequence identity+sequence similarity) can be determined using any homology comparison software computing a pairwise sequence alignment. As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89 (22): 10915-9]

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “reaction mixture,” or “cell-free reaction mixture” or “recombinant cell-free reaction mixture” as used herein, refers to a solution containing reagents necessary to carry out a given reaction. A cell-free expression system “reaction mixture” or “reaction solution” typically contains a crude or partially-purified extract, (such as from a bacteria, plant cell, microalgae, fungi, or mammalian cell) nucleotide translation template, and a suitable reaction buffer for promoting cell-free protein synthesis from the translation template. In one aspect, the CF reaction mixture can include an exogenous RNA translation template. In other aspects, the CF reaction mixture can include a DNA expression template encoding an open reading frame operably linked to a promoter element for a DNA-dependent RNA polymerase. In these other aspects, the CF reaction mixture can also include a DNA-dependent RNA polymerase to direct transcription of an RNA translation template encoding the open reading frame. In these other aspects, additional NTPs and divalent cation cofactor can be included in the CF reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention. Moreover, those of ordinary skill will understand that some components in a reaction mixture, while utilized in certain embodiments, are not necessary to generate cell-free expression products. The term “cell-free expression products” may be any biological product produced through a cell-free expression system.

As used herein, the term “transformation” or “genetically modified” refers to the transfer of one or more nucleic acid molecule(s) into a cell, preferably through an expression vector. A microorganism is “transformed” or “genetically modified” by a nucleic acid molecule transduced into the bacteria or cell or organism when the nucleic acid molecule becomes stably replicated. As used herein, the term “transformation” or “genetically modified” encompasses all techniques by which a nucleic acid molecule can be introduced into a cell or organism, such as a bacteria.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor or binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

An “expression vector” is nucleic acid capable of replicating in a selected host cell or organism, or in vitro environment, such as a cell-free expression system or other IVT system. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). The terms “expression product” as it relates to a protein expressed in a cell-free expression system as generally described herein, are used interchangeably and refer generally to any peptide or protein having more than about 5 amino acids. The polypeptides may be homologous to, or may be exogenous, meaning that they are heterologous, i.e., foreign, to the organism from which the cell-free extract is derived, such as a human protein, plant protein, viral protein, yeast protein, etc., produced in the cell-free extract.

In some embodiments, the term a nucleic acid or peptide may be from a source, such as a virus. In this context a “derived” nucleic acid or peptide means extracted from, or expressed and isolated from a bacteria, eukaryotic cell or other source. For example, in one embodiment a capping protein may be derived from an expression vector expressed in a bacteria, or eukaryotic cell.

As used herein, “modified nucleotides” (including references to modified NTP, modified ATP, modified GTP, modified CTP, and modified UTP) refers to any noncanonical nucleoside, nucleotide or corresponding phosphorylated versions thereof. Modified nucleotides may include one or more backbone or base modifications. Examples of modified nucleotides include d1, dU, 8-oxo-dG, dX, and THF. Additional examples of modified nucleotides include the modified nucleotides disclosed in U.S. Patent Publication Nos. US20170056528A1, US20160038612A1, US2015/0167017A1, and US20200040026A1. Modified nucleotides may include naturally or non-naturally occurring nucleotides.

As used herein, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′-end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative). All publications, patents, and patent applications mentioned in this specification are herein incorporated by reference to the same extent as if each individual publication, patent, or patent application was specifically and individually indicated to be incorporated by reference.

As used herein, “RNA sample” or “sample” refers to a composition that may or may not comprise a target RNA. For example, an RNA sample may be known to include or suspected of including such target RNA and/or an RNA sample may be a composition to be evaluated for the presence of a target RNA. An RNA sample may comprise a naturally occurring target RNA (e.g., extracted from a cell, tissue, or organism), a target RNA produced by in vitro transcription, and/or a chemically synthesized RNA.

As used herein, “single uncapped RNA target species” refers to a mixture of target RNA molecules that have essentially the same sequence. Transcripts made by in vitro transcription and RNA oligonucleotides made by solid-phase synthesis are examples single uncapped RNA target species. It is recognized that a certain amount of the RNA products in such a mixture may be truncated. Single uncapped RNA target species may sometimes contain modified nucleotides (e.g., noncanonical nucleotides that are not found in nature). Preparations of RNA from a cell contain a complex mixture of naturally occurring RNA molecules having different sequences; such preparations do not contain only targeted uncapped RNA species but also contain a wide variety of non-target RNAs. In some embodiments, the targeted uncapped RNA species is a single species of RNA.

As used herein, “target RNA” refers to a polyribonucleotide of interest. A polyribonucleotide may be or comprise a therapeutic RNA or precursor thereof (e.g., an uncapped precursor of a capped therapeutic RNA). A target RNA may arise from cellular transcription or in vitro transcription. A target RNA may be present in a mixture, for example, an in vitro transcription reaction mixture, a cell, or a cell lysate. A target RNA may be uncapped. If desired or required, a target RNA may be contacted with a decapping enzyme, for example, as a co-treatment with or pre-treatment before capping.

As used herein, “uncapped” refers to an RNA (a) that does not have a cap and (b) that can be used as a substrate for a capping enzyme. Uncapped RNA typically has a tri- or di-phosphorylated 5′-end. RNAs transcribed in vitro have a triphosphate group at the 5′-end.

As used herein, “variant” refers to a protein that has an amino acid sequence that is different from a naturally occurring amino acid sequence (i.e., having less than 100% sequence identity to the amino acid sequence of a naturally occurring protein) but that is at least 80%, at least 85%, at least 90%, at least 95%, at least 97%, at least 98% or at least 99% identical to the naturally occurring amino acid sequence.

As used herein, “fragment” refers to a portion of a peptide or nucleotide sequence that still retains the activity of the whole.

As used herein, a “bioreactor” may be any form of enclosed apparatus configured to maintain an environment conducive to the production of macromolecules in vitro. A bioreactor may be configured to run on a batch, continuous, or semi-continuous basis, for example by a feeder reaction solution. Examples of a bioreactor and conditions for synthesis of RNA or other macromolecules has been previously described in by Koglin, et al. PCT/US2021/027774

TABLE 1
Characteristics of His-Tagged Capping Enzyme

| Position | 1-1193 | |
|---|---|---|
| Summary | MANK . . . HHHH 1193 Aas | |
| Molecular Weight | 138751.30 | Da |
| Isoelectric Point (pl) | 6.67 | |
| Extinction Coefficent | | |
| Cys fully reduced | 163200.00 | M−1 cm−1 |
| Abs 0.1% (1 g/l) | 1.176 | |
| Cys fully oxidized | 163575.00 | M−1 cm−1 |

| | |
|---|---|
| Abs 0.1% (1 g/l) | 1.179 |
| Instability Index | 33.10 (stable) |

TABLE 2
Sequence Homology Between SEQ ID NO. 1 and 2
93.6% identity in 1185 residues overlap; Score: 5907.0; Gap frequency: 0.0%

| | | |
|---|---|---|
| UserSeq1 | 1 | MANKIKEQNIFDDVLTDEQYTNIDTMIKDFKKSKDMELEVSFRNISYASYMRISEHLANI |
| UserSeq2 | 1 | MANKIKEHNIFDDILTDEQYTKIDTMIKDFKKSNDMELEVSFRNISYASYMRISEHLVNI |
| | | ******* ************* *********** *********************** ** |
| UserSeq1 | 61 | VDENDISAQDSLDISIMLADNNTYRVSILDVGEIEKFIQRFSRSRPDEIQKYIISLKPSD |
| UserSeq2 | 61 | VNENDISAQDSLDISIILVDNNTYRVSILDVDKIENFIQRFSRSRPDEIQKHILGLKSSD |
| | | * ************** * ************ ** **************** * ** ** |
| UserSeq1 | 121 | DIEIMYKDRGSANRLYIDDFGTVFKLTRETPITKENSKPDLNGNEKMLYRYKSRYSFNIN |
| UserSeq2 | 121 | DVELMFKDRGSANRLYIDDFGTVFKLTRETPVTKENSKPNVTGNEKMLYRYKSRYSFNMN |
| | | * * * ************************* ******* **************** * |
| UserSeq1 | 181 | ENVRIDITNVQESTNLWNLPRRHSNYEIEIEVVNKNIKIETLLSEVESVLKIIQDSDIPI |
| UserSeq2 | 181 | ENVRIDITDVQESTNLWNLPRRHSNYEIEIEVVNKNIKIETLLAEVESVLKIIQDSDIPV |
| | | ******** ********************************** *************** |
| UserSeq1 | 241 | GKKEASEVIQKYQTLLNVKGSHIDSRNVVSIETQHIVKFIPNKYAITDKADGERYFLFST |
| UserSeq2 | 241 | GKKEANEVIQKYQSLLNVKGSHIDSRNVVSIETQHIVKFIPNKYAITDKADGERYFLFST |
| | | ***** ******* ********************************************** |
| UserSeq1 | 301 | QDGVYLLSTNMVVKKLGIEINDKKFQNMLLDGELIKNEHGYMFLAFDVIYANGVDYRYND |
| UserSeq2 | 301 | EDGVYLLSTNMVVKKIDIEINDKKFQNMLLDGELIKNEHGYMFLAFDVIYANGIDYRYND |
| | | ************** ************************************ ****** |
| UserSeq1 | 361 | KYILTHRLTVLNNIIDQCFGTLIPFTDYTDKNADLEIDKIKSFYGKELKSYWTAFIKEMK |
| UserSeq2 | 361 | KYILTHRLTVLNNIIDQCFGTLIPFTDYTDKNSDLEIDKIKTFYNKELKSYWAAFIKEMN |
| | | ******************************** ******** ** ******* ****** |
| UserSeq1 | 421 | KSDGLFVSRKLYFVPYGIDSSEVFMYADMVWKLSVYSKLTPYKLDGIIYTPINTPYMIKV |
| UserSeq2 | 421 | KSNGLFVSRKLYFVPYGIDSSEVFMYADMVWKLSVYSKLTPYKLDGIIYTPINTPYMIKV |
| | | ** ********************************************************* |
| UserSeq1 | 481 | SPENLDTMPMEYKWKTPAQNSIDFYIKFEKDAKGNDAIYYDNAVVRADANAYKICTLHVG |
| UserSeq2 | 481 | SPENLDTVPMEYKWKTPMQNSIDFYIKFEKDAKGNDAIFYDNAVVRADANAYKICTLHVG |
| | | ******* ********* ******************** ********************* |
| UserSeq1 | 541 | INRGGQEKPVPFKVNGAEQRANIYVTDGEATDLEGNVINDETVVEFIFDITKPDIDDAYK |
| UserSeq2 | 541 | INRGGQEKPVPFKVNGVEQRANIYITDGEATDLDGNVINDETVVEFIFDTTKPDIDDAYK |
| | | **************** ******* ******** *************** ********** |
| UserSeq1 | 601 | WIPLRTRYDKTESVQKYGKKYGNNLNIALRIWRTIINPITEENISTLGNPSTFQKEIDRM |
| UserSeq2 | 601 | WTPIRTRYDKTESVQKYGKKYGNNLNIALRIWRTIINPITEENIATLGNPSTFQKEIDRM |
| | | * * **************************************** *************** |
| UserSeq1 | 661 | SKTVDVYNKQSFVYYQKKTGNAAGMRAFNNWIKSNMILTYCTGKHNVLDIGCGRGGDLIK |
| UserSeq2 | 661 | SKSVEAYNKQSFVYYQKKTGNAAGMRAFNNWIKSNMILTYCTGKHNVLDIGCGRGGDLIK |
| | | ** * ***************************************************** |
| UserSeq1 | 721 | FIYAGIDEYVGIDIDHNGLYVISDSAYNRYKNLRNTHKHIPQMYFIHADARARFNVKSQE |
| UserSeq2 | 721 | FIYAGIDEYVGIDIDHNGLYVISDSAYNRYKNLRNTHKHVPQMYFIHADARARFNVESQE |
| | | *************************************** **************** *** |
| UserSeq1 | 781 | SVLSNMTAFNKKLIETHLSGNKKYNVINAQFTLHYYLSDELSWSNFCKNINDHLETNGYL |
| UserSeq2 | 781 | SVLPNMTPFNKKLIETHLSGNKKYNVINAQFTLHYYLSDELSWSNFCKNINDHLETNGYF |
| | | *** *** *************************************************** |
| UserSeq1 | 841 | LVTAFDGKLIYDRLSGKQKMTVSYTDNNGNKNIFFEINKVYNDNDKNFGVGMAIDLYNSL |
| UserSeq2 | 841 | LVTAFDGKLIYDRLLGKQKMTVAYTDNNGNKNIFFEINKVYSDNDKNFGVGIAIDLYNSL |
| | | ************** ******* ****************** ********* ******** |
| UserSeq1 | 901 | ISNPGTYIREYLVFPEFLEQSLKKNCGLELVESDSFFNLFNLYKNYFTEEDMGEFSISDT |
| UserSeq2 | 901 | ISNPGTYIREYLVFPEFLEKSLKKNCGLELVESDSFFNLFNLYKNYFTEEDMGEFSISDT |
| | | ******************* **************************************** |
| UserSeq1 | 961 | SAKRHDEIRNFYLSLKPNNHSDVETDAALASFKFSMLNRYYVFKKTTKIDVIEPSRIVGM |
| UserSeq2 | 961 | SAKRHEEIRNFYLSLKPNNHTDVEMDAALASFKFSMLNRYYVFKKTTKIDVIEPSRIVGM |
| | | ***** ************** *** *********************************** |
| UserSeq1 | 1021 | NHKINLGKVLTPYFHTNKMIIDPAKKSSQINKIYHAIRKRYAPVKPSVYLIRHTIPEDIV |
| UserSeq2 | 1021 | NHKINLGKVLTPYFHTNKMIIDPAKKSSQINKIYHAIRKKYTPIKPTVYLIRHTIPEDIV |
| | | **************************************** ** ** ************* |
| UserSeq1 | 1081 | DNEVYRRNKLEFSKIKDGPDGKTLLIYKSPDKYFYPVYYQNNVYKDHDEFLQYRLSTKNT |
| UserSeq2 | 1081 | DNEVYRRNKLEFSKIKDGPDDKTLLIYKSPDKYFYPVYYQNNVYKDHDEFLQYRLSTKNT |
| | | ******************** *************************************** |
| UserSeq1 | 1141 | KGITYDSDDKYEKSGTYLLESNKIVNDLDVLVALSEKLKINRNSD |
| UserSeq2 | 1141 | KGITYDSDNKYEKSGTYLLDSNKIVNDLDILVALTEKIKINRDSN |
| | | ******** ********** ********* **** ** **** * |

What is claimed is:

1. A method for capping RNA in vitro comprising the steps:
generating an RNA sample comprising an uncapped target RNA and a buffering agent; and
contacting said RNA sample comprising an uncapped target RNA with an RNA guanylyltransferase capping enzyme from a Tupanviruses virus, or a fragment or variant thereof, under conditions wherein a Cap-0 RNA is synthesized in vitro.

2. The method of claim 1, wherein said capping enzyme comprises a capping enzyme from Tupanvirus soda lake virus or Tupanvirus deep ocean virus.

3. The method of claim 2, wherein said capping enzyme from Tupanvirus soda lake virus comprises a capping enzyme according to the amino acid sequence SEQ ID NO. 1, or a fragment or variant thereof, or wherein said capping enzyme from Tupanvirus deep ocean virus comprises a capping enzyme according to the amino acid sequence SEQ ID NO. 2, or a fragment or variant thereof.

4. The method of claim 1, wherein said capping enzyme comprises a capping enzyme according to the amino acid sequence SEQ ID NO. 1, or a sequence at is at least 80%-99% sequence identity to SEQ ID NO. 1.

5. The method of claim 1, wherein said step of contacting comprises contacting guanosine triphosphate (GTP) or modified GTP.

6. The method of claim 1, wherein said step of contacting comprises a contacting a buffering agent.

7. The method of claim 1, and further comprising the step of methylating the Cap-0 RNA forming a Cap-1 RNA.

8. The method of claim 7, wherein said step of methylating comprises contacting said Cap-0 RNA with a methyltransferase and a methyl donor.

9. The method of claim 8, wherein said methyl donor comprises S-adenosyl methionine (SAM).

10. The method of claim 8, wherein said methyltransferase comprises guanine-7-methyltransferase.

11. The method of claim 1, wherein said ratio of RNA guanylyltransferase capping enzyme and uncapped RNA is between 1:50 and 1:1, or between 1:100 and 50:1.

12. The method of claim 1, wherein said step of contacting comprises contacting at a temperature between 30° and 50° C., or between 20° and 60° C.

13. The method of claim 1, further comprising synthesizing the uncapped RNA using solid-phase oligonucleotide synthesis chemistry.

14. The method of claim 1, further comprising synthesizing the uncapped RNA by contacting a DNA template encoding the uncapped RNA and a polymerase to produce the uncapped RNA.

15. The method of claim 1, wherein the uncapped target RNA comprises one or more pseudouridines.

16. The method of claim 1, wherein said step of contacting comprises contacting in an in vitro expression system.

17. The method of claim 16, wherein said in vitro expression system comprises a cell-free expression system.

18. A composition comprising:
an uncapped target RNA; and
a RNA guanylyltransferase capping enzyme from a Tupanviruses virus, or a fragment or variant thereof;
guanosine triphosphate (GTP); and
a buffering agent.

19. The composition of claim 18, wherein said capping enzyme comprises a capping enzyme from Tupanvirus soda lake virus or Tupanvirus deep ocean virus.

20. The composition of claim 19, wherein said capping enzyme from Tupanvirus soda lake virus comprises a capping enzyme according to the amino acid sequence SEQ ID NO. 1, or a fragment or variant thereof, or wherein said capping enzyme from Tupanvirus deep ocean virus comprises a capping enzyme according to the amino acid sequence SEQ ID NO. 2, or a fragment or variant thereof.

21. The composition of claim 18, wherein said capping enzyme comprises a capping enzyme according to the amino acid sequence SEQ ID NO. 1, or a sequence at is at least 80%-99% sequence identity to SEQ ID NO. 1.

22. The composition of claim 18, and further comprising a polymerase and ribonucleotides, for transcribing a template polynucleotide encoding a uncapped target RNA.

23. The composition of claim 22 wherein said polymerase comprises a T7 bacteriophage polymerase.

24. The composition of claim 18, and further comprising a methyltransferase and a methyl donor.

25. The composition of claim 24, wherein said methyl donor comprises S-adenosyl methionine (SAM).

26. The composition of claim 24, wherein said methyltransferase comprises guanine-7-methyltransferase.

27. The composition of claim 24, wherein said methyltransferase comprises 2′-O-methyltransferase enzyme (2′OMTase).

28. The composition of claim 18, wherein said ratio of RNA guanylyltransferase capping enzyme and uncapped RNA is between 1:50 and 1:1, or between 1:100 and 50:1.

29. The composition of claim 18, wherein said composition has a temperature between 30° and 50° C., or between 20° and 60° C.

30. The composition of claim 18, wherein said uncapped RNA is synthesized using solid-phase oligonucleotide synthesis chemistry.

31. The composition of claim 18, wherein said uncapped RNA is synthesized by contacting a DNA template encoding the uncapped RNA and a polymerase to produce the uncapped RNA.

32. The composition of claim 18, wherein the uncapped target RNA comprises one or more pseudouridines.

33. The composition of claim 18, wherein the composition is RNase-free and optionally comprises one or more RNase inhibitors.

34. A system for producing capped RNA transcripts in vitro comprising:
an RNA guanylyltransferase capping enzyme from a Tupanviruses virus, or a fragment or variant thereof, wherein the enzyme is in a storage buffering agent; and
a reaction buffering agent.

35. The system of claim 34, further comprises a polymerase and ribonucleotides, for transcribing a template polynucleotide encoding a uncapped target RNA.

36. The system of claim 34, further comprises guanosine triphosphate (GTP) or modified GTP.

37. The system of claim 34, wherein said polymerase comprises a T7 bacteriophage polymerase.

38. The system of claim 34, further comprises a methyl donor or a methyltransferase enzyme, or both methyl donor and methyltransferase enzyme.

39. The system of claim 38, wherein said methyl donor comprises S-adenosyl methionine (SAM), and said methyltransferase enzyme comprises cap 2′-O-methyltransferase enzyme (2′OMTase).

40. The system of claim 34, wherein said wherein said RNA guanylyltransferase capping enzyme comprises a capping enzyme from Tupanvirus soda lake virus or Tupanvirus deep ocean virus.

41. The system of claim 34, wherein said capping enzyme from Tupanvirus soda lake virus comprises a capping enzyme according to the amino acid sequence SEQ ID NO. 1, or a fragment or variant thereof, or wherein said capping enzyme from Tupanvirus deep ocean virus comprises a capping enzyme according to the amino acid sequence SEQ ID NO. 2, or a fragment or variant thereof.

42. The system of claim 34, wherein said RNA guanylyltransferase capping enzyme wherein said capping enzyme comprises a capping enzyme according to the amino acid sequence SEQ ID NO. 1, or a sequence at is at least 80%-99% sequence identity to SEQ ID NO. 1.

43. The system of claim 34, wherein the buffering agents are RNase-free and optionally comprises one or more RNase inhibitors.

44. A system for producing capped RNA transcripts in a cell-free expression system comprising:
a reaction mixture having cell-free reaction components necessary for in vitro macromolecule synthesis, including an RNA guanylyltransferase capping enzyme from a Tupanviruses virus, or a fragment or variant thereof; and
a storage buffering agent.

45. The system of claim 44, further comprises a polymerase and ribonucleotides, for transcribing a template polynucleotide encoding a uncapped target RNA.

46. The system of claim 45, wherein said polymerase comprises a T7 bacteriophage polymerase.

47. The system of claim 44, further comprises guanosine triphosphate (GTP) or modified GTP.

48. The system of claim 44, further comprises a methyl donor or a methyltransferase enzyme, or both methyl donor and methyltransferase enzyme.

49. The system of claim 48, wherein said methyl donor comprises S-adenosyl methionine (SAM), and said methyltransferase enzyme comprises cap 2′-O-methyltransferase enzyme (2′OMTase).

50. The system of claim 44, wherein said wherein said RNA guanylyltransferase capping enzyme comprises a capping enzyme from Tupanvirus soda lake virus or Tupanvirus deep ocean virus.

51. The system of claim 44, wherein said capping enzyme from Tupanvirus soda lake virus comprises a capping enzyme according to the amino acid sequence SEQ ID NO. 1, or a fragment or variant thereof, or wherein said capping enzyme from Tupanvirus deep ocean virus comprises a capping enzyme according to the amino acid sequence SEQ ID NO. 2, or a fragment or variant thereof.

52. The system of claim 44, wherein said RNA guanylyltransferase capping enzyme wherein said capping enzyme comprises a capping enzyme according to the amino acid sequence SEQ ID NO. 1, or a sequence at is at least 80%-99% sequence identity to SEQ ID NO. 1.

53. The system of claim 44, wherein the buffering agent and/or reaction mixture are RNase-free and optionally comprises one or more RNase inhibitors.

54. An RNA capping enzyme comprising an amino acid sequence that is at least 90% identical to SEQ ID NO: 1 or 2.

55. An RNA capping enzyme comprising an amino acid sequence that is at least 80% identical to SEQ ID NO: 1 or 2.

56. An expression vector having a nucleotide sequence, operably linked to a promoter, encoding an RNA capping enzyme comprising an amino acid sequence that is at least 90% identical to SEQ ID NO: 1 or 2.

57. An expression vector having a nucleotide sequence, operably linked to a promoter, encoding an RNA capping enzyme comprising an amino acid sequence that is at least 80% identical to SEQ ID NO: 1 or 2.

58. An RNA capping enzyme comprising an amino acid sequence according to SEQ ID NO: 1, or 2.

59. An RNA capping enzyme comprising an amino acid sequence according to SEQ ID NO: 1 or 2.

60. An expression vector having a nucleotide sequence, operably linked to a promoter, encoding an RNA capping enzyme comprising an amino acid sequence according to SEQ ID NO: 1 or 2.

61. An expression vector having a nucleotide sequence, operably linked to a promoter, encoding an RNA capping enzyme comprising an amino acid sequence according to SEQ ID NO: 1 or 2.

62. An isolated RNA capping enzyme having an amino acid sequence according to SEQ ID NO:
1 or 2.

63. An isolated RNA capping enzyme having an amino acid sequence that is at least 80% identical to SEQ ID NO: 1 or 2.

64. An isolated RNA capping enzyme having an amino acid sequence that is at least 90% identical to SEQ ID NO: 1 or 2.

65. An isolated RNA capping enzyme having an amino acid sequence that is at least 98% identical to SEQ ID NO: 1 or 2.

66. An isolated RNA capping enzyme having an amino acid sequence that is at least 99% identical to SEQ ID NO: 1 or 2.

67. A kit for producing capped RNA transcripts in a cell-free expression system comprising:
a container comprising a reaction mixture having cell-free reaction components necessary for in vitro macromolecule synthesis, including an RNA guanylyltransferase capping enzyme from a Tupanviruses virus, or a fragment or variant thereof; and
a storage buffering agent; and
instructions for use.

68. The kit of claim 67, further comprises a polymerase and ribonucleotides, for transcribing a template polynucleotide encoding a uncapped target RNA.

69. The kit of claim 68, wherein said polymerase comprises a T7 bacteriophage polymerase.

70. The kit of claim 67, further comprises guanosine triphosphate (GTP) or modified GTP.

71. The kit of claim 67, further comprises a methyl donor or a methyltransferase enzyme, or both methyl donor and methyltransferase enzyme.

72. The kit of claim 71, wherein said methyl donor comprises S-adenosyl methionine (SAM), and said methyltransferase enzyme comprises cap 2′-O-methyltransferase enzyme (2′OMTase).

73. The kit of claim 67, wherein said wherein said RNA guanylyltransferase capping enzyme comprises a capping enzyme from Tupanvirus soda lake virus or Tupanvirus deep ocean virus.

74. The kit of claim 67, wherein said capping enzyme from Tupanvirus soda lake virus comprises a capping enzyme according to the amino acid sequence SEQ ID NO. 1, or a fragment or variant thereof, or wherein said capping enzyme from Tupanvirus deep ocean virus comprises a capping enzyme according to the amino acid sequence SEQ ID NO. 2, or a fragment or variant thereof.

75. The kit of claim 67, wherein said RNA guanylyltransferase capping enzyme wherein said capping enzyme comprises a capping enzyme according to the amino acid sequence SEQ ID NO. 1, or a sequence at is at least 80%-99% sequence identity to SEQ ID NO. 1.

76. The kit of claim 67, wherein the buffering agent and/or reaction mixture are RNase-free and optionally comprises one or more RNase inhibitors.


SEQUENCE LISTING (3 sequences)

SEQ ID NO: 1 - protein, 1185 aa, Tupanvirus
MANKIKEQNIFDDVLTDEQYTNIDTMIKDFKKSKDMELEVSFRNISYASYMRISEHLANIVDENDISAQDSLDISIMLADNNTYRVSILDVGEIEKFIQRFSRSRPDEIQKYIISLKPSDDIEIMYKDRGSANRLYIDDFGTVFKLTRETPITKENSKPDLNGNEKMLYRYKSRYSFNINENVRIDITNVQESTNLWNLPRRHSNYEIEIEVVNKNIKIETLLSEVESVLKIIQDSDIPIGKKEASEVIQKYQTLLNVKGSHIDSRNVVSIETQHIVKFIPNKYAITDKADGERYFLFSTQDGVYLLSTNMVVKKLGIEINDKKFQNMLLDGELIKNEHGYMFLAFDVIYANGVDYRYNDKYILTHRLTVLNNIIDQCFGTLIPFTDYTDKNADLEIDKIKSFYGKELKSYWTAFIKEMKKSDGLFVSRKLYFVPYGIDSSEVFMYADMVWKLSVYSKLTPYKLDGIIYTPINTPYMIKVSPENLDTMPMEYKWKTPAQNSIDFYIKFEKDAKGNDAIYYDNAVVRADANAYKICTLHVGINRGGQEKPVPFKVNGAEQRANIYVTDGEATDLEGNVINDETVVEFIFDITKPDIDDAYKWIPLRTRYDKTESVQKYGKKYGNNLNIALRIWRTIINPITEENISTLGNPSTFQKEIDRMSKTVDVYNKQSFVYYQKKTGNAAGMRAFNNWIKSNMILTYCTGKHNVLDIGCGRGGDLIKFIYAGIDEYVGIDIDHNGLYVISDSAYNRYKNLRNTHKHIPQMYFIHADARARFNVKSQESVLSNMTAFNKKLIETHLSGNKKYNVINAQFTLHYYLSDELSWSNFCKNINDHLETNGYLLVTAFDGKLIYDRLSGKQKMTVSYTDNNGNKNIFFEINKVYNDNDKNFGVGMAIDLYNSLISNPGTYIREYLVFPEFLEQSLKKNCGLELVESDSFFNLFNLYKNYFTEEDMGEFSISDTSAKRHDEIRNFYLSLKPNNHSDVETDAALASFKFSMLNRYYVFKKTTKIDVIEPSRIVGMNHKINLGKVLTPYFHTNKMIIDPAKKSSQINKIYHAIRKRYAPVKPSVYLIRHTIPEDIVDNEVYRRNKLEFSKIKDGPDGKTLLIYKSPDKYFYPVYYQNNVYKDHDEFLQYRLSTKNTKGITYDSDDKYEKSGTYLLESNKIVNDLDVLVALSEKLKINRNSD

SEQ ID NO: 2 - protein, 1185 aa, Tupanvirus
MANKIKEHNIFDDILTDEQYTKIDTMIKDFKKSNDMELEVSFRNISYASYMRISEHLVNIVNENDISAQDSLDISIILVDNNTYRVSILDVDKIENFIQRFSRSRPDEIQKHILGLKSSDDVELMFKDRGSANRLYIDDFGTVFKLTRETPVTKENSKPNVTGNEKMLYRYKSRYSFNMNENVRIDITDVQESTNLWNLPRRHSNYEIEIEVVNKNIKIETLLAEVESVLKIIQDSDIPVGKKEANEVIQKYQSLLNVKGSHIDSRNVVSIETQHIVKFIPNKYAITDKADGERYFLFSTEDGVYLLSTNMVVKKIDIEINDKKFQNMLLDGELIKNEHGYMFLAFDVIYANGIDYRYNDKYILTHRLTVLNNIIDQCFGTLIPFTDYTDKNSDLEIDKIKTFYNKELKSYWAAFIKEMNKSNGLFVSRKLYFVPYGIDSSEVFMYADMVWKLSVYSKLTPYKLDGIIYTPINTPYMIKVSPENLDTVPMEYKWKTPMQNSIDFYIKFEKDAKGNDAIFYDNAVVRADANAYKICTLHVGINRGGQEKPVPFKVNGVEQRANIYITDGEATDLDGNVINDETVVEFIFDTTKPDIDDAYKWTPIRTRYDKTESVQKYGKKYGNNLNIALRIWRTIINPITEENIATLGNPSTFQKEIDRMSKSVEAYNKQSFVYYQKKTGNAAGMRAFNNWIKSNMILTYCTGKHNVLDIGCGRGGDLIKFIYAGIDEYVGIDIDHNGLYVISDSAYNRYKNLRNTHKHVPQMYFIHADARARFNVESQESVLPNMTPFNKKLIETHLSGNKKYNVINAQFTLHYYLSDELSWSNFCKNINDHLETNGYFLVTAFDGKLIYDRLLGKQKMTVAYTDNNGNKNIFFEINKVYSDNDKNFGVGIAIDLYNSLISNPGTYIREYLVFPEFLEKSLKKNCGLELVESDSFFNLFNLYKNYFTEEDMGEFSISDTSAKRHEEIRNFYLSLKPNNHTDVEMDAALASFKFSMLNRYYVFKKTTKIDVIEPSRIVGMNHKINLGKVLTPYFHTNKMIIDPAKKSSQINKIYHAIRKKYTPIKPTVYLIRHTIPEDIVDNEVYRRNKLEFSKIKDGPDDKTLLIYKSPDKYFYPVYYQNNVYKDHDEFLQYRLSTKNTKGITYDSDNKYEKSGTYLLDSNKIVNDLDILVALTEKIKINRDSN

SEQ ID NO: 3 - protein, 1193 aa, Tupanvirus
MANKIKEQNIFDDVLTDEQYTNIDTMIKDFKKSKDMELEVSFRNISYASYMRISEHLANIVDENDISAQDSLDISIMLADNNTYRVSILDVGEIEKFIQRFSRSRPDEIQKYIISLKPSDDIEIMYKDRGSANRLYIDDFGTVFKLTRETPITKENSKPDLNGNEKMLYRYKSRYSFNINENVRIDITNVQESTNLWNLPRRHSNYEIEIEVVNKNIKIETLLSEVESVLKIIQDSDIPIGKKEASEVIQKYQTLLNVKGSHIDSRNVVSIETQHIVKFIPNKYAITDKADGERYFLFSTQDGVYLLSTNMVVKKLGIEINDKKFQNMLLDGELIKNEHGYMFLAFDVIYANGVDYRYNDKYILTHRLTVLNNIIDQCFGTLIPFTDYTDKNADLEIDKIKSFYGKELKSYWTAFIKEMKKSDGLFVSRKLYFVPYGIDSSEVFMYADMVWKLSVYSKLTPYKLDGIIYTPINTPYMIKVSPENLDTMPMEYKWKTPAQNSIDFYIKFEKDAKGNDAIYYDNAVVRADANAYKICTLHVGINRGGQEKPVPFKVNGAEQRANIYVTDGEATDLEGNVINDETVVEFIFDITKPDIDDAYKWIPLRTRYDKTESVQKYGKKYGNNLNIALRIWRTIINPITEENISTLGNPSTFQKEIDRMSKTVDVYNKQSFVYYQKKTGNAAGMRAFNNWIKSNMILTYCTGKHNVLDIGCGRGGDLIKFIYAGIDEYVGIDIDHNGLYVISDSAYNRYKNLRNTHKHIPQMYFIHADARARFNVKSQESVLSNMTAFNKKLIETHLSGNKKYNVINAQFTLHYYLSDELSWSNFCKNINDHLETNGYLLVTAFDGKLIYDRLSGKQKMTVSYTDNNGNKNIFFEINKVYNDNDKNFGVGMAIDLYNSLISNPGTYIREYLVFPEFLEQSLKKNCGLELVESDSFFNLFNLYKNYFTEEDMGEFSISDTSAKRHDEIRNFYLSLKPNNHSDVETDAALASFKFSMLNRYYVFKKTTKIDVIEPSRIVGMNHKINLGKVLTPYFHTNKMIIDPAKKSSQINKIYHAIRKRYAPVKPSVYLIRHTIPEDIVDNEVYRRNKLEFSKIKDGPDGKTLLIYKSPDKYFYPVYYQNNVYKDHDEFLQYRLSTKNTKGITYDSDDKYEKSGTYLLESNKIVNDLDVLVALSEKLKINRNSDLEHHHHHH